(19) United States
(12) Patent Application Publication
(10) Pub. No.: US 2020/0282222 A1
(43) Pub. Date: Sep. 10, 2020

(54) IMPLANTABLE MEDICAL DEVICE WITH BULK METALLIC GLASS ENCLOSURE

(71) Applicant: LIQUIDMETAL TECHNOLOGIES, INC., Lake Forest, CA (US)

(72) Inventor: Joseph W. Stevick, Lake Forest, CA (US)

(21) Appl. No.: 16/782,592

(22) Filed: Feb. 5, 2020

Related U.S. Application Data
(60) Provisional application No. 62/801,811, filed on Feb. 6, 2019.

(51) Int. Cl. A61N 1/375; A61N 1/39; A61N 1/36

(52) U.S. Cl. CPC A61N 1/3758; A61N 1/36071; A61N 1/3968; A61N 1/37512

(57) ABSTRACT

An enclosure for an implantable cardiac or neurostimulation device includes a bulk metallic glass alloy. In some arrangements, the enclosure is configured to house one or more components of an implantable pacemaker. In some arrangements, the enclosure is configured to house one or more components of an implantable defibrillator.

### CROSS-REFERENCE TO RELATED PATENT APPLICATION

This application claims the benefit of and priority to U.S. Provisional Application No. 62/801,811, filed Feb. 6, 2019, the entire disclosure of which is hereby incorporated by reference herein.

### BACKGROUND

The present disclosure relates generally to the field of medical device materials and methods of manufacturing. More specifically, the present disclosure relates to amorphous biocompatible materials and their related processing techniques applied to enclosure structures for implantable medical devices such as pacemakers, defibrillators, stimulators, cochlear implants, and other types of implantable medical devices.

### SUMMARY

One embodiment relates to an enclosure for an implantable cardiac or neuro stimulation device. The enclosure includes a bulk metallic glass alloy. The enclosure may be configured to house one or more components of an implantable pacemaker or the implantable defibrillator.

Another embodiment relates to an implantable stimulation device. The implantable stimulation device includes one or more electrodes, a pulse generator configured to generate and deliver, via the one or more electrodes, stimulation therapy to a patient, and an enclosure configured to house at least the pulse generator. The enclosure is at least partially fabricated from a bulk metallic glass alloy and is configured for long-term implantation within a patient.

According to an exemplary embodiment, the stimulation therapy may be heart pacing therapy. According to other exemplary embodiments, the stimulation therapy may be cardioversion defibrillation therapy. According to yet still other exemplary embodiments, the stimulation therapy may be pain treatment therapy.

In some embodiments, the bulk metallic glass alloy is an alloy of at least zirconium, titanium, copper, nickel, and aluminum. In some embodiments, the enclosure includes two or more pieces configured to snap, screw, or precisely mate together to form the enclosure. In some embodiments, the enclosure includes at least one retaining clip and/or support feature configured to lock at least one of the pulse generator, battery, wires, or other component of the implantable medical device in place inside the enclosure. In some embodiments, the enclosure is an injection-molded component. Similarly, such internal features can be designed to physically separate various internal components from one another for assembly purposes or other design considerations (such as isolating a battery from other internal components).

Another embodiment relates to a method of manufacturing an enclosure for an implantable medical device such as a cardiac or neurostimulation device. The method includes providing one or more molds for the enclosure and injection molding the enclosure, using the one or more molds, from a bulk metallic glass alloy. The finished enclosure is configured to house one or more components of the implantable medical device and is configured for long-term implantation within a patient.

This summary is illustrative only and is not intended to be in any way limiting. Other aspects, inventive features, and advantages of the devices and/or processes described herein, will become apparent in the detailed description set forth herein, taken in conjunction with the accompanying figures, wherein like reference numerals refer to like elements.

### BRIEF DESCRIPTION OF THE FIGURES

FIG. 1 illustrates a schematic of a time-temperature transformation (TTT) diagram for an exemplary bulk solidifying amorphous alloy.

FIG. 2 illustrates an implantable stimulation device, according to an exemplary embodiment.

FIG. 3 illustrates results of salt fog corrosion testing of medical device materials, including a bulk metallic glass, according to an exemplary embodiment.

FIG. 4 illustrates skin depth versus electromagnetic radiation frequency for medical device materials, including a bulk metallic glass, according to an exemplary embodiment.

### DETAILED DESCRIPTION

Before turning to the figures, which illustrate the exemplary embodiments in detail, it should be understood that the present disclosure is not limited to the details or methodology set forth in the description or illustrated in the figures. It should also be understood that the terminology used herein is for the purpose of description only and should not be regarded as limiting.

Implantable medical devices often include a biocompatible metallic enclosure. For example, many medical devices are enclosed in a surgical stainless steel (e.g., 316L or 316LVM stainless steel) or titanium alloy (e.g., Grade 5 titanium alloy) housing. However, existing metallic enclosures for medical devices may include constraints, such as a narrow range of elastic deformation that may limit how and in what form the enclosures may be fabricated. As such, other metallic enclosure options may be desirable for certain medical device applications.

Referring generally to the figures, medical device enclosures made of an amorphous metal alloy such as a bulk metallic glass (BMG) are provided. Material scientists have known about the existence and potential of bulk metallic glass alloys for several decades, but large-scale commercialization of such materials has been a relatively recent undertaking. These materials are known by many different names, including but not limited to: bulk amorphous metals, glassy metals, Vitreloy™, Liquidmetal™, bulk-solidifying amorphous alloys, bulk amorphous alloys, etc. BMGs are a class of materials categorized by their ability to be fabricated with uniquely disorganized atomic structures in thicknesses typically greater than 1 mm. This does not preclude fabricating structures less thick than 1 mm but indicates that the alloy is capable of existing in structures greater than 1 mm thick. In the long history of metal alloys, BMGs are the first to exhibit no periodic structure in their atomic arrangement. They are instead composed of unique and carefully engineered proportions of dissimilar atoms that solidify and cool from a molten state while retaining amorphous, non-crystalline (e.g., glassy or liquid-like) structures down to room temperatures and below. It is precisely this amorphous structure that gives a BMG its unique and advantageous physical properties. Oft-cited BMG properties are strength, strength-to-weight, hardness, elastic limit, resistance to corrosion, electromagnetic properties, and precision. An important property of a BMG alloy, and one that has been improved by material scientists over the past few decades, is its critical cooling rate: the slowest rate at which the material can be quenched (from a liquid to a solid) while maintaining the enabling amorphous atomic structure. Amorphous alloys can have many superior properties over their crystalline counterparts as mentioned earlier.

Alloys described herein can be amorphous or substantially amorphous. The material structure of a BMG may result in low shrinkage during cooling and resistance to plastic deformation. The absence of grain boundaries (e.g., two-dimensional defects in the crystalline lattice), which may be weak spots in crystalline materials in some cases, may lead to better resistance to wear and corrosion in amorphous alloys. In one embodiment, amorphous metals, while considered glasses, may also be much tougher and less brittle than oxide glasses and ceramics. Scientific literature is rich with additional detailed information about BMG materials, designs, properties, and industrial potentials.

A measure of how “amorphous” an amorphous alloy may be includes amorphicity. For example, a composition can be partially amorphous, substantially amorphous, or completely amorphous. Amorphicity can be measured in terms of a degree of crystallinity. For example, in one embodiment, an alloy having a low degree of crystallinity can be said to have a high degree of amorphicity. In one embodiment, for example, an alloy having 60 vol % crystalline phase can have a 40 vol % amorphous phase. The amorphous and crystalline phases can have the same chemical composition and differ only in the microstructure—e.g., one amorphous and the other crystalline. Microstructure in one embodiment includes the structure of a material as revealed by a microscope at, for example, 25× magnification or higher. Alternatively, the two phases can have different chemical compositions and microstructures. Either way, these mixed microstructure BMG alloys can be intentionally created and are often referred to as composites. Their advantages can include cosmetic appearances, enhanced ductility, lower cost, etc. The non-amorphous phase can be a crystal or a plurality of crystals. The crystals can be in the form of particulates of any shape, such as spherical, ellipsoid, wire-like, rod-like, sheet-like, flake-like, or irregular shape. In one embodiment, crystals can have a dendritic form. For example, an at least partially amorphous composite composition can have a crystalline phase in the shape of dendrites dispersed in an amorphous phase matrix; the dispersion can be uniform or non-uniform, and the amorphous phase and the crystalline phase can have the same or a different chemical composition. In one embodiment, the amorphous phase and the crystalline phase have substantially the same chemical composition. In another embodiment, the crystalline phase can be more ductile than the BMG phase.

FIG. 1 shows the time-temperature-transformation (TTT) cooling curve of an exemplary bulk solidifying amorphous alloy, or TTT diagram. Bulk-solidifying amorphous metals do not experience a liquid/solid crystallization transformation upon cooling, as with conventional metals. Instead, the highly fluid, non-crystalline form of the metal found at high temperatures (near a “melting temperature” Tₘ) becomes more viscous as the temperature is reduced (near a “glass transition temperature” T_{g}), eventually taking on the outward physical properties of a conventional solid. In some embodiments, Tₓ and T_{g} are determined from standard differential scanning calorimeter (DSC) measurements at typical heating rates (e.g., 20° C./min) as the onset of crystallization temperature and the onset of glass transition temperature.

With regard to thermoplastic forming operations, the supercooled liquid region (the temperature region between T_{g} and Tₓ) is a manifestation of the extraordinary stability against crystallization of bulk solidification alloys. In this temperature region the bulk solidifying alloy can exist as a high viscous liquid. The viscosity of the bulk solidifying alloy in the supercooled liquid region can vary between 10¹² Pa·s at the glass transition temperature down to 10⁵ Pa·s at the crystallization temperature, the high temperature limit of the supercooled liquid region. Liquids with such viscosities can undergo substantial plastic strain under an applied pressure. As such, various embodiments herein make use of the large plastic formability or thermoplastic formability in the supercooled liquid region in forming, joining, molding, and separating parts.

The schematic TTT diagram of FIG. 1 shows processing methods of die casting from, at, or above Tₘ to below T_{g} without the time-temperature trajectory (shown as (1) as an example trajectory) hitting the TTT curve. During die casting, the forming takes place substantially simultaneously with fast cooling to avoid the trajectory hitting the TTT curve. The processing methods for superplastic forming (SPF) may also occur from, at, or below T_{g} to below Tₘ without the time-temperature trajectory (shown as (2), (3), and (4) as example trajectories) hitting the TTT curve. In SPF, the amorphous BMG is reheated into the supercooled liquid region where the available processing window could be much larger than die casting, resulting in better controllability of the process. The SPF process does not require fast cooling to avoid crystallization during cooling. Also, as shown by example trajectories (2), (3), and (4), the SPF can be carried out with the highest temperature during SPF being above Tₙₒₛₑ or below Tₙₒₛₑ, up to about Tₘ. If one heats up a piece of amorphous alloy but manages to avoid hitting the TTT curve, one will have heated “between T_{g} and Tₘ,” but one will not have reached Tₓ.

The methods described herein can be applicable to any type of amorphous alloy, whether that alloy is based on zirconium, iron, nickel, titanium, copper, platinum, gold, or another element. For example, an amorphous alloy may be based on zirconium with additional elements in smaller mass or weight percentages. Regardless of the base element, an amorphous alloy can include any other elements such as zirconium, hafnium, titanium, copper, nickel, platinum, palladium, iron, magnesium (Mg), gold, lanthanum (La), silver, aluminum (Al), molybdenum, niobium, beryllium (Be), yttrium (Yt), or combinations thereof. Namely, the alloy can include any combination of elements such as these in its chemical formula or chemical composition. The elements can be present at different weight or volume percentages. For example, an “iron-based” alloy can refer to an alloy having a non-insignificant weight percentage of iron present therein. The weight percent can be, for example, at least about 20 wt %, such as at least about 40 wt %, such as at least about 50 wt %, such as at least about 60 wt %, or such as at least about 80 wt %.

For example, in many commercial applications today, the amorphous alloy can be based on zirconium and can have the formula (Zr, Ti)ₐ(Ni, Cu, Fe)_{b}(Be, Al, Si, B)_{c}, wherein a, b, and c each represents a weight or atomic percentage. In one embodiment, a is in the range of from 30 to 75, b is in the range of from 5 to 60, and c is in the range of from 0 to 50 in atomic percentages. Alternatively, in some embodiments, the amorphous alloy can have the formula (Zr, Ti)ₐ(Ni, Cu)_{b}(Be)_{c}, wherein a, b, and c each represents a weight or atomic percentage. In one embodiment, a is in the range of from 40 to 75, b is in the range of from 5 to 50, and c is in the range of from 5 to 50 in atomic percentages. The alloy can also have the formula (Zr, Ti)ₐ(Ni, Cu)_{b}(Be)_{c}, wherein a, b, and c each represents a weight or atomic percentage. In one embodiment, a is in the range of from 45 to 65, b is in the range of from 7.5 to 35, and c is in the range of from 10 to 37.5 in atomic percentages. Alternatively, in some embodiments, the alloy can have the formula (Zr)ₐ(Nb, Ti)_{b}(Ni, Cu)_{c}(Al)_{d}, wherein a, b, c, and d each represents a weight or atomic percentage. In one embodiment, a is in the range of from 45 to 65, b is in the range of from 0 to 10, c is in the range of from 20 to 40, and d is in the range of from 7.5 to 15 in atomic percentages. One exemplary embodiment of the aforedescribed alloy system is a Zr—Cu—Ti—Ni—Al based amorphous alloy Liquidmetal™, such as LM105 and LM106a, as fabricated by Materion and injection-molded into commercial products by Liquidmetal Technologies, CA, USA. Some additional examples of zirconium- and non-zirconium-based amorphous alloys of the different systems are provided in Table 1 and Table 2.

TABLE 1
Exemplary amorphous alloy compositions

| | Atm | Atm | Atm | Atm | Atm | Atm | Atm | Atm |
|---|---|---|---|---|---|---|---|---|
| Alloy | % | % | % | % | % | % | % | % |
| 1 | Fe | Mo | Ni | Cr | P | C | B | |
| | 68.00% | 5.00% | 5.00% | 2.00% | 12.50% | 5.00% | 2.50% | |
| 2 | Fe | Mo | Ni | Cr | P | C | B | Si |
| | 68.00% | 5.00% | 5.00% | 2.00% | 11.00% | 5.00% | 2.50% | 1.50% |
| 3 | Pd | Cu | Co | P | | | | |
| | 44.48% | 32.35% | 4.05% | 19.11% | | | | |
| 4 | Pd | Ag | Si | P | | | | |
| | 77.50% | 6.00% | 9.00% | 7.50% | | | | |
| 5 | Pd | Ag | Si | P | Ge | | | |
| | 79.00% | 3.50% | 9.50% | 6.00% | 2.00% | | | |
| 6 | Pt | Cu | Ag | P | B | Si | | |
| | 74.70% | 1.50% | 0.30% | 18.0% | 4.00% | 1.50% | | |

TABLE 2
Additional exemplary amorphous alloy compositions (atomic %)

| Alloy | Atm % | Atm % | Atm % | Atm % | Atm % | Atm % |
|---|---|---|---|---|---|---|
| 1 | Zr | Ti | Cu | Ni | Be | |
| | 41.20% | 13.80% | 12.50% | 10.00% | 22.50% | |
| 2 | Zr | Ti | Cu | Ni | Be | |
| | 44.00% | 11.00% | 10.00% | 10.00% | 25.00% | |
| 3 | Zr | Ti | Cu | Ni | Nb | Be |
| | 56.25% | 11.25% | 6.88% | 5.63% | 7.50% | 12.50% |
| 4 | Zr | Ti | Cu | Ni | Al | Be |
| | 64.75% | 5.60% | 14.90% | 11.15% | 2.60% | 1.00% |
| 5 | Zr | Ti | Cu | Ni | Al | |
| | 52.50% | 5.00% | 17.90% | 14.60% | 10.00% | |
| 6 | Zr | Nb | Cu | Ni | Al | |
| | 57.00% | 5.00% | 15.40% | 12.60% | 10.00% | |
| 7 | Zr | Cu | Ni | Al | | |
| | 50.75% | 36.23% | 4.03% | 9.00% | | |
| 8 | Zr | Ti | Cu | Ni | Be | |
| | 46.75% | 8.25% | 7.50% | 10.00% | 27.50% | |
| 9 | Zr | Ti | Ni | Be | | |
| | 21.67% | 43.33% | 7.50% | 27.50% | | |
| 10 | Zr | Ti | Cu | Be | | |
| | 35.00% | 30.00% | 7.50% | 27.50% | | |
| 11 | Zr | Ti | Co | Be | | |
| | 35.00% | 30.00% | 6.00% | 29.00% | | |
| 12 | Zr | Ti | Fe | Be | | |
| | 35.00% | 30.00% | 2.00% | 33.00% | | |
| 13 | Au | Ag | Pd | Cu | Si | |
| | 49.00% | 5.50% | 2.30% | 26.90% | 16.30% | |
| 14 | Au | Ag | Pd | Cu | Si | |
| | 50.90% | 3.00% | 2.30% | 27.80% | 16.00% | |
| 15 | Pt | Cu | Ni | P | | |
| | 57.50% | 14.70% | 5.30% | 22.50% | | |
| 16 | Zr | Ti | Nb | Cu | Be | |
| | 36.60% | 31.40% | 7.00% | 5.90% | 19.10% | |
| 17 | Zr | Ti | Nb | Cu | Be | |
| | 38.30% | 32.90% | 7.30% | 6.20% | 15.30% | |
| 18 | Zr | Ti | Nb | Cu | Be | |
| | 39.60% | 33.90% | 7.60% | 6.40% | 12.50% | |
| 19 | Cu | Ti | Zr | Ni | | |
| | 47.00% | 34.00% | 11.00% | 8.00% | | |
| 20 | Zr | Co | Al | | | |
| | 55.00% | 25.00% | 20.00% | | | |

The aforedescribed amorphous alloy systems can further include additional elements, such as additional transition metal elements, including yttrium, niobium, chromium, vanadium, and cobalt. The additional elements can be present at less than or equal to about 30 wt %, such as less than or equal to about 20 wt %, such as less than or equal to about 10 wt %, or such as less than or equal to about 5 wt %. In one embodiment, the additional, optional element(s) is/are at least one of cobalt, manganese, zirconium, tantalum, niobium, tungsten, yttrium, titanium, vanadium and hafnium, which may cause the alloy system to form carbides and further improve wear and corrosion resistance. Further optional elements may include phosphorous, germanium and arsenic (e.g., totaling up to about 2 wt % and, in some embodiments, less than 1 wt %) to reduce the melting point. Otherwise incidental impurities should be, in some embodiments, less than about 2 wt % and preferably 0.5 wt %.

As noted above, an amorphous metal alloy such a BMG may be used to create an enclosure for a medical device. In particular, BMG enclosures may possess a number of advantages when used as implantable medical device enclosures. For example, in various embodiments and as described in further detail below, BMGs may have a high strength-to-weight ratio, which allows devices to remain lightweight but robust against damage; have electromagnetic properties appropriate for magnetic resonance imaging (MRI) safety; and allow for good transmission of electromagnetic emissions for communication and/or wireless charging (e.g., at 402-405 MHz). As also discussed in further detail below, in various embodiments, BMGs exhibit excellent corrosion resistance for prolonged exposure to body fluid environments with minimal degradation of material properties or leaching of ions, as well as implantable-grade biocompatibility test results.

BMGs also exhibit many properties that are advantageous for manufacturing medical device enclosures. Because BMGs exhibit glass-like properties, BMG enclosures may be created through injection molding manufacturing to create complex, irregular, precise, and/or small-scale geometries in large volumes at low cost. Moreover, BMG enclosures may be manufactured through the use of computer numerical control (CNC) of machine tools, for example, to create stamped-type housings. Further, BMGs have a larger elastic range of deformation than some medical device materials (e.g., 2% elastic range), which allows BMG enclosures to be manufactured with snap-type assembly configurations without loss of mechanical integrity (e.g., plastic deformation of the enclosures) during the assembly process. Alternatively, BMG medical device enclosures may be manufactured with a potential assembly/sealing process for bonding to similar or dissimilar materials.

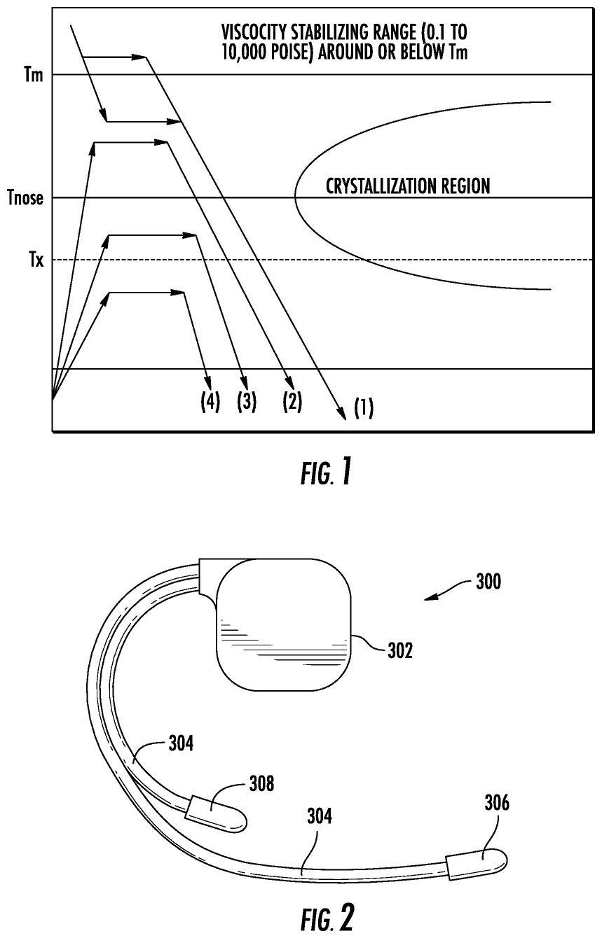

Referring to FIG. 2, an implantable stimulation device 300 is shown, according to an exemplary embodiment. The stimulation device 300 is configured to provide stimulation to a patient. For example, the stimulation device 300 may be a pacemaker, a defibrillator, a stimulator, etc. The stimulation device 300 includes an enclosure 302 housing electrical components of the stimulation device 300, such as a battery, a control system, and a pulse generator. The stimulation device 300 also includes a number of leads 304 coupled to the enclosure 302. In the embodiment of FIG. 2, the stimulation device 300 includes two leads 304. However, it should be understood that, in other embodiments, the stimulation device 300 may include a different number of leads 304 (e.g., based on the application of the stimulation device 300). As an illustration, if the stimulation device 300 is used to provide pacing stimulation to a right atrial node and a right ventricular node, the stimulation device 300 may include a right atrial lead and a right ventricular lead and one or more sensing leads.

Each of the leads 304 is configured to provide stimulation to a patient and/or sense one or more physiological signals of a patient. In the embodiment shown in FIG. 2, a first lead 304 ends in an electrode array 306, and a second lead 304 ends in a sensor 308. The electrode array 306 includes one or more electrodes configured to deliver stimulation therapy (e.g., pacing therapy, defibrillation therapy, etc.), generated by a pulse generator housed in the enclosure 302, to the patient. The sensor 308 is configured to sense one or more physiological signals of the patient. For example, the sensor 308 may sense electrical activity of the patient's heart, which the control system uses to determine whether and when stimulation therapy should be delivered to the patient.

As an illustration, in some embodiments, the sensor 308 is anchored in a patient's right atrium (e.g., such that the sensor can sense electrical activity in the patient's sinoatrial node and atrioventricular node), and the electrode array 306 is anchored in a patient's right ventricle (e.g., at or near the apex of the right ventricle). The sensor 308 gathers data on electrical activity in the patient's heart. A control system housed in the enclosure 302 determines, based on the electrical activity, whether and when the stimulation device 300 should provide stimulation to the patient. For example, if the stimulation device 300 is a pacemaker, the control system determines whether the patient's heart is experiencing an arrhythmia (e.g., the patient is experiencing bradycardia or tachycardia). In response to determining that the patient's heartbeat is irregular, the control system causes the pulse generator to generate and deliver, via the electrode array 306, pacing stimulation therapy to the heart to restore the patient's regular heartbeat. As another example, if the stimulation device 300 is a defibrillator, the control system determines whether the patient's heart is experiencing fibrillation (e.g., the patient is experiencing ventricular fibrillation). In response to determining that the patient is experiencing fibrillation, the control system causes the pulse generator to generate and deliver, via the electrode array 306, cardioversion-defibrillation stimulation therapy to treat the fibrillation.

As another illustration, in some embodiments, the stimulation device 300 is a stimulator configured to provide electrical stimulation to a nerve system of a patient. For example, the stimulation device 300 may be configured to provide stimulation to treat pain, to regulate the patient's heart rate, etc. Accordingly, in such embodiments, the electrode array 306 is configured to be implanted near a nerve of the patient. Additionally, in such embodiments, the stimulation device 300 may not include the sensor 308. As an example, the stimulation device 300 may be a stimulator configured to provide pain treatment stimulation therapy to a patient's spinal cord. The control system may be configured to receive a signal from an external system (e.g., a handheld programming device) instructing the control system to begin providing stimulation. In response, the control system causes the pulse generator to generate and deliver, via the electrode array 306, stimulation signals to the patient's spinal cord to treat the patient's pain.

It should be understood that the configuration of the leads 304, the electrode array 306, and the sensor 308 is intended to be exemplary and that other configurations may be used in other embodiments. For example, while the embodiment of the stimulation device 300 includes multiple leads 304, in some embodiments, the enclosure 302 may instead be configured such that few or no leads 304 are required. For example, the stimulation device 300 may be a leadless pacing device, with pacing stimulation and sensing capabilities provided by one or more electrodes integrated into the enclosure 302. Alternatively, in some embodiments, the stimulation device 300 may not include one or more separate sensing leads. Instead, the stimulation electrodes may also serve as sensing electrodes, or the sensing electrodes may be provided on the same lead(s) as the stimulation electrodes.

In various embodiments, as discussed above, the enclosure 302 of the stimulation device 300 may be partially or entirely formed of an amorphous metal alloy such as a BMG. For example, the enclosure 302 may be mostly formed of a BMG, with a small plastic piece housing the location where the leads 304 extend from the enclosure 302. Fabricating the enclosure 302 from a BMG may provide the enclosure 302 with the desirable properties of BMGs discussed above, including, for example, a high strength-to-weight ratio; MRI safety; good transmission of electromagnetic emissions for remote charging, wireless communication between the device and another device outside the patient's body, or other purposes; and the ability to be created through injection molding, with snap-type or threaded assemblies, and/or with a potential assembly/sealing processing for bonding to other materials.

Additionally, fabricating the enclosure 302 from a BMG may provide the enclosure 302 with desirable biocompatibility. As an illustration, pre-clinical materials testing has shown that pacemaker enclosures formed from LM105, a BMG with the composition Zr₅₂₅Ti₅Cu₁₇₉Ni_{14.6}Al₁₀ by atomic weight, produced by Liquidmetal Technologies, possess a number of biocompatible properties, as described in more detail below.

To begin with, a first round of testing was performed on as-molded LM105 specimens based on parts 4, 5, 10, and 11 of the International Organization for Standardization (ISO) 10993 test methods for medical devices. The first round of testing examined the basic biocompatibility of the as-molded LM105 specimens. ISO 10993-4 testing includes hemocompatibility testing (e.g., testing for the rupturing of red blood cells and release of cytoplasm into blood plasma in response to the tested material). The ISO 10993-4 testing included four sets of tests.

A hemolysis test (based on American Standard for Testing and Materials (ASTM) F756) was conducted on the LM105 specimens. An extraction of the LM105 specimens was immersed in phosphate buffered solution mixed with blood solution. National Committee for Clinical Laboratory Standards (NCCLS) cardiac magnetic resonance (CMR) imaging was then performed to measure the hemolysis of the phosphate buffered solution mixed with the blood solution in response to the extraction. In particular, the hemolysis test measured hemoglobin concentration compared to a negative reference control. The LM105 specimens showed a 0.5% concentration above the negative reference control (pass).

Complement activation testing was conducted on the LM105 specimens, in particular, a C3a assay and SC5b-9 assay. The complement activation testing measured the ability of the material to trigger complement activation: C3a for anaphylotoxicity and SC5b-9 for cell lysis (e.g., showing tissue breakdown). The test articles were exposed to normal human serum (NHS), and an extraction was plated into triplicate wells of C3a and SC5b-9 plates. The LM105 specimens showed 0.38% activation for the C3a assay and 0% activation for the SC5b-9 assay (pass).

A partial thromboplastin time (PTT) test and a prothrombin time (PT) test were conducted on the LM105 specimens to measure the ability of the material to cause clot formation (e.g., showing activation of the intrinsic coagulation pathway for the PTT test and activation of the extrinsic pathway for the PT test). For each of these tests, an extraction of the LM105 specimens was exposed to human plasma. For the PTT test, the LM105 specimens showed 32.3% (97 sec.) and 46.1% (138 sec.) of negative plasma control (moderate activation), and for the PT test, the LM105 specimens showed 13 seconds of clotting time and less than a 2-fold increase in PT (pass). As such, the LM105 specimens passed the hemocompability testing as non-hemolytic.

The ISO 10993-5 testing examined the cytotoxicity (e.g., cellular toxicity) of the tested material. For this testing, in vitro cytotoxicity tests were conducted, specifically to examine MEM elution, which correlates with a material's toxicity to cells. Additionally, an extraction of the LM105 specimens was immersed in cell culture and plated onto L-929 fibroblast cells to test whether the specimens caused cell lysis or inhibited cell growth. For these cytotoxicity tests, the LM105 enclosures were shown to be non-cytotoxic, with a cytotoxicity grade of 0 at 24, 48, and 72 hours.

The ISO 10993-10 testing examined sensitization and irritation of the material. For sensitization, a guinea pig (GP) maximization test was conducted to determine the skin sensitization of the LM105 specimens (e.g., their ability to cause an allergic response) and elicitation of contact dermatitis. Accordingly, intradermal and topical induction of specimen extracts were performed on guinea pigs. The result of the GP maximization test was a score of 0 for 24 and 48 hours, showing that the materials were non-sensitizing. For irritation, an intracutaneous reactivity test was performed to test the ability of the material to cause intracutaneous irritation (e.g., by the effect of toxic leachables). For this test, an extraction of the LM105 specimens was injected into guinea pigs with observation after 24, 48, and 72 hours. The result was 0.2 for polar and 0.3 for non-polar extractions, which showed that the LM105 specimens were non-irritating.

The ISO 10993-11 testing examined the systemic toxicity (e.g., the effect on the system from absorption and distribution of a toxicant) of the LM105 specimens. In particular, an acute systemic toxicity test was performed through single exposure with a 72-hour observation period. The result was no effect on test subjects with no abnormalities and no weight loss, showing that the LM105 specimens were non-systemic-toxic. In summary, the results from the ISO 10993-4, 10993-5, 10993-10, and 10993-11 testing suggested that LM105 in the as-molded condition is a potential candidate for surface contact, blood contact, and implantation, and thus is a potential candidate for implantable pacemaker, fibrillation, stimulation, etc. devices as described above.

Long-term implant-specific testing was also performed on deburred and passivated LM105 injection molded parts. These tests were selected from parts 3, 6, 10, and 11 for ISO10993. The ISO10993-3 testing examined the genotoxicity, carcinogenicity, and reproductive toxicity of the LM105 specimens. The testing included a mouse lymphoma assay based on 4- and 24-hour treatments. The results showed that the LM105 enclosures were non-mutagenic, as the mutant frequency was less than 90×10⁻⁶ of the average mutant frequency of the concurrent negative control. As utilized herein, “long-term implantation” means a contact duration in a body of a human for at least 30 days or 720 hours.

Passivation is a process by which the already corrosion-resistant surface of a zirconium-based BMG part can be further enhanced to diminish any risk of forming oxidides or leaching ions into the body. The fact that alloys such as LM105 can be commercially passivated as part of the BMG component manufacturing process is extremely valuable to the pacemaker application since resistance to the body fluid environment is critical for the performance of an enclosure device, and a more extended low-risk device lifetime is inherently advantageous for any implantable device.

The ISO10993-6 and 10993-10 testing evaluated the effects of chronic exposure post-implantation. In particular, the ISO 10993-6 testing examined sub-chronic systemic toxicity 90 days after a test article was implanted in the test subject. Tests were conducted for local effects after the implantation to evaluate organ weight changes, gross necropsy findings, and histopathy results from organs and tissues. No abnormalities were noted at necropsy. All the implanted sites appeared within normal limits, and there were no signs of local and/or general toxicity (pass). The ISO 10993-10 testing evaluated the biological response to chronic exposure of an implanted test article. Devices were implanted in rabbits with paraffin histoprocessing; tests were conducted for irritation and skin sensitization. The final test article score was 0.3, and the test article was determined to be a non-irritant to the subject's tissue as compared to the negative control sample (pass). As such, based on the ISO 10993-6 and 10993-10 testing, the LM150 specimens were determined to be non-irritants.

The ISO 10993-11 testing was performed to evaluate systemic toxicity. Two sets of tests were performed. (1) Materials mediated pyrogenicity tests were performed according to tests for systemic toxicity from the United States Pharmocopeia (USP)<151> Pyrogen Test Regulatory Standards. The <151> Pyrogen Test Regulatory Standards tests provide general information on the detection of material mediated pyrgenicity of the test article under investigation. Based on the results of this study, the LM105 test article showed no evidence of material mediated pyrogenicity (pass). (2) Sub-chronic systemic toxicity based on an implant after 90 days was tested for local effects after implantation. Similar to the ISO 10993-6 tests discussed above, these tests evaluate organ weight changes, gross necropsy findings, and histopathy results from organs and tissues. No abnormalities were noted at necropsy, all the implanted sites appeared within normal limits, and there were no signs of local and/or general toxicity (pass). As such, the results of the ISO 10993-6 showed that the LM105 specimens were non-systemic-toxic. Accordingly, the ISO 10993-3, 10993-6, 10993-10, and 10993-11 tests discussed above showed that LM105 parts have the appropriate biocompatibility to be considered for implantable pacemaker enclosure applications, as well as applications for other implantable medical devices.

However, even if potential implantation materials show good biocompatibility, leaching of metal ions into a body fluid environment is also a potential roadblock for implantation of the materials. Because LM105 contains nickel as one of its constituent elements, which can leach into a body fluid environment, the LM105 enclosures were tested for nickel release in a simulated body fluid environment. These tests were conducted according to the EN 1811:2011 test method (which is used to test nickel release in the European Union). The test involves placing the article in an artificial sweat solution for one week and then measuring the nickel in the solution by atomic absorption spectroscopy or inductively coupled plasma mass spectrometry (ICP-MS). The article is then given a pass or fail grading. Both LM105 as-molded and LM105 that had been blasted and passivated were tested. The tests for the LM105 as-molded were all below the measurable limit, while the tests for the blasted and passivated LM105 were 0.0048 or less. These test results indicate that the nickel release rates for as-molded LM105 are below the limit standards for prolonged and piercing body contact, further suggesting that LM105 enclosures would be safe for human implantation.

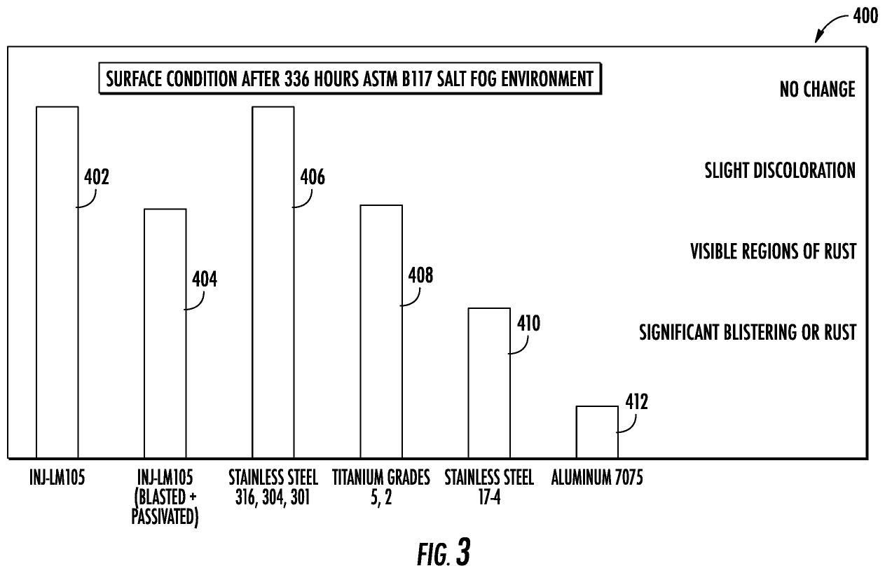

Salt fog corrosion tests were also conducted on the LM105 test coupons for 336 hours in a salt fog environment according to ASTM test standard B117. The results of the as-molded LM105 are shown in chart 400 illustrated in FIG. 3 (shown as bar 402). Chart 400 also includes the results for blasted and passivated LM105 (shown as bar 404) and other metals used in medical devices, including stainless steels 316, 304, 301 (shown as bar 406), titanium Grades 5, 2 (shown as bar 408), stainless steel 17-4 (shown as bar 410), and aluminum 7075 (shown as bar 412). As shown by bar 402, the as-molded LM105 showed minimum discoloration and no corrosion on LM105 test coupons. This resistance is equal or better than high grade stainless steels and titanium alloys, which are commonly used for long-term human implantation. Additional testing showed no change in LM105 test coupons after over 1,000 hours in the same environment.

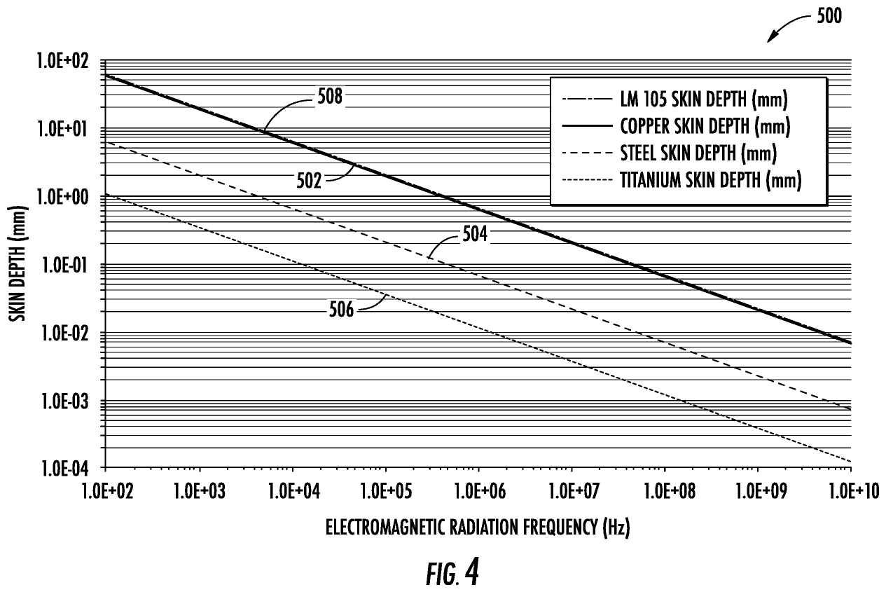

Additionally, LM105 enclosures are geared towards non-ferrous behavior for safety and compatibility in MRI environments. The electromagnetic properties of LM105 make it an MRI-safe material (e.g., the LM105 shows no attractive or repulsive forces in a static B-field), and minimal artifacts are produced around the implant site when imaged using MRI due to the low conductivity and low magnetic susceptibility of the LM105, which is close to the relative magnetic susceptibility of air. For example, FIG. 4 illustrates a graph 500 of the skin depth (mm) versus the electromagnetic radiation frequency (Hz) for LM105, (shown as line 502), copper (shown as line 504), steel (shown as line 506), and titanium (shown as line 508). As illustrated in FIG. 4, the calculated skin depth of injection molded LM105 is very similar to that of titanium, which has been shown to generate fewer (e.g., less intense) artifacts than stainless steel alloys (e.g., as shown by Knott et al., “A Comparison of Magnetic and Radiographic Imaging Artifact After Using Three Types of Metal Rods: Stainless Steel, Titanium, and Vitallium,” Spine Journal, Vol. 10, p. 789-794 (2010)). The skin depth calculations indicate the LM105 would have similar “transparency” to electromagnetic signals used for remote communication or power supply charging. The operational band for these devices is around 4×10⁸ Hz, at which the skin depth is about 0.03 mm for both titanium and LM105, as illustrated in FIG. 4. This indicates that LM105 pacemaker enclosures would have a similar electromagnetic response to MRI environments as well as communication signals and provide comparable image quality as titanium, while maintaining other, advantageous properties over titanium (e.g., in fabrication flexibility).

Accordingly, as shown by the above-discussed testing, BMGs like LM105 may be a good candidate for enclosures used for implantable medical devices, as well as for other medical device components. Moreover, due to the glass-like nature of LM105 and the larger range of recoverable elastic strain that BMGs such as LM105 can experience, creating pacemaker enclosures out of LM105 may allow for more freedom of design, compact size, and anatomically-favorable geometries over traditional medical device materials (e.g., anatomically-matched geometries rather than geometries dictated by manufacturing methods such as stamping or machining). As such, according to some embodiments, LM105 enclosures may have a number of production advantages, including faster production cycle times with fewer production steps/stages necessary, dimensional repeatability with high yield processes, and allowing for more complex but repeatable geometric features at a reduced cost.

As an illustration, BMGs often have a larger elastic range than many metals and metal alloys (e.g., LM105 may show around 2% recoverable elastic strain). As such, potential BMG pacemaker enclosure designs can incorporate advantageous elastic features not possible with, for example, titanium alloys. For instance, in some embodiments, a BMG pacemaker enclosure may include interlocking snap-fit features for precise and repeatable alignment of the two halves of the pacemaker during assembly. Utilizing the 2% recoverable elastic strain of the BMG alloy, these features can be locked and unlocked multiple cycles while providing the same amount of locking force each time (e.g., without showing plastic deformation). In one embodiment, these same features can be used to align and lock internal components into place with retaining clips or other support features that are monolithic with the pacemaker enclosure body. For example, these retaining clips or other support features may be used to lock at least one of a pulse generator, battery, wires, or other component of an implantable medical device inside the enclosure. Similarly, support features can be designed to physically separate various internal components from one another for assembly purposes or for design purposes, such as to physically separate the battery of an implantable medical device from other internal components. Alternatively, in another embodiment, the entire pacemaker enclosure rims could be designed to snap together (e.g., similar to a plastic Easter egg). Welding could then be used at these enclosure mating locations to prevent unlocking in some implementations. In other embodiments, potential BMG enclosure designs may include a different type of interlocking fit (e.g., for precisely mating the two enclosure halves together), such as threading for screwing the two halves of the enclosure together.

As another example, while titanium is often used in implantable medical device enclosures due to its biocompability, the cost of manufacturing high grade titanium enclosures can be significant. As shown by the above-described testing, LM105 enclosures may show similar or better biocompatibility than titanium and can be produced more inexpensively Similar conclusions may be reached for other BMG medical device enclosures, such as LM105 defibrillator enclosures and LM105 stimulation enclosures.

Additionally, while embodiments of medical devices fabricated from amorphous alloys such as BMGs are described above with reference to stimulation devices, such as pacemakers, defibrillators, and stimulators, the biocompatible features and advantageous fabrication properties of BMGs may be leveraged in other medical device applications. For example, implantable or external pumps for drugs, cerebrospinal fluid (CSF), and other fluids; cochlear implants; power sources; microelectronics packages inside other devices; high voltage capacitors for treating tachycardia and other physiological conditions; and arterial blood pressure measurement systems may be at least partially fabricated of amorphous alloys, including BMGs.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

The term “coupled,” as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other with a separate intervening member and any additional intermediate members coupled with one another, or with the two members coupled to each other with an intervening member that is integrally formed as a single unitary body with one of the two members. Such members may be coupled mechanically, electrically, and/or fluidly.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below,” etc.) are merely used to describe the orientation of various elements in the FIGURES. It should be noted that the orientation of various elements may differ according to other exemplary embodiments, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

What is claimed is:

1. An enclosure for an implantable cardiac or neuro stimulation device comprising a bulk metallic glass alloy.

2. The enclosure of claim 1, wherein the bulk metallic glass alloy is an alloy of at least zirconium, titanium, copper, nickel, and aluminum.

3. The enclosure of claim 1, wherein the enclosure comprises two or more pieces configured to snap together to form the enclosure.

4. The enclosure of claim 1, wherein the enclosure comprises at least one retaining clip configured to lock at least one component of the implantable cardiac or neuro stimulation device in place inside the enclosure.

5. The enclosure of claim 1, wherein the enclosure is an injection-molded component.

6. The enclosure of claim 1, wherein the enclosure is configured to house one or more components of an implantable pacemaker.

7. The enclosure of claim 1, wherein the enclosure is configured to house one or more components of an implantable defibrillator.

8. An implantable stimulation device, comprising:
one or more electrodes;
a pulse generator configured to generate and deliver, via the one or more electrodes, stimulation therapy to a patient; and
an enclosure configured to house at least the pulse generator, wherein the enclosure is at least partially fabricated from a bulk metallic glass alloy and is configured for long-term implantation within the patient.

9. The implantable stimulation device of claim 8, wherein the bulk metallic glass alloy is an alloy of at least zirconium, titanium, copper, nickel, and aluminum.

10. The implantable stimulation device of claim 8, wherein the enclosure comprises two or more pieces configured to snap together to form the enclosure.

11. The implantable stimulation device of claim 8, wherein the enclosure comprises at least one retaining clip configured to lock at least one of the pulse generator or another component of the implantable stimulation device in place inside the enclosure.

12. The implantable stimulation device of claim 8, wherein the enclosure is an injection-molded component.

13. The implantable stimulation device of claim 8, wherein the stimulation therapy is heart pacing therapy.

14. The implantable stimulation device of claim 8, wherein the stimulation therapy is cardioversion defibrillation therapy.

15. The implantable stimulation device of claim 8, wherein the stimulation therapy is pain treatment therapy.

16. A method of manufacturing an enclosure for an implantable cardiac or neuro stimulation device, the method comprising:
providing one or more molds for the enclosure; and
injection molding the enclosure, using the one or more molds, from a bulk metallic glass alloy;
wherein a finished enclosure is configured to house one or more components of the implantable cardiac or neuro stimulation device and is configured for long-term implantation within a patient.

17. The method of claim 16, wherein the bulk metallic glass alloy is an alloy of at least zirconium, titanium, copper, nickel, and aluminum.

18. The method of claim 16, wherein the one or more molds are configured to produce two or more pieces configured to snap together to form the enclosure.

19. The method of claim 16, wherein the enclosure is configured to house one or more components of an implantable pacemaker.

20. The method of claim 16, wherein the enclosure is configured to house one or more components of an implantable defibrillator.
